(19) United States
(12) Patent Application Publication
(10) Pub. No.: US 2003/0050202 A1
(43) Pub. Date: Mar. 13, 2003

(54) Kit for making transparent soap at home and method for making transparent soap using the same

(76) Inventor: Byeng-Ro An, Seoul (KR)

Correspondence Address: HARRISON & EGBERT, 412 MAIN STREET, 7TH FLOOR, HOUSTON, TX, 77002, US

(21) Appl. No.: 10/177,848

(22) Filed: Jun. 24, 2002

(30) Foreign Application Priority Data
Sep. 7, 2001 (KR) 2001-55004

(51) Int. Cl. A61K 7/50

(52) U.S. Cl. 510/141; 510/147

(57) ABSTRACT

Disclosed is a kit for making transparent soaps at home and a method for making transparent soaps using the kit, which can be used to conveniently make transparent soaps having various shapes at one time at home. The kit includes an upper plate and a sidewall extending downward from the entire edge of the upper plate. The upper plate has a plurality of mould cavities opened upward. The method includes the steps of preparing a transparent soap composition, melting the transparent soap composition, injecting the molten composition to the kit, solidifying the molten composition and taking the solidified soap out of the kit.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates to a kit for making transparent soaps at home and a method for making transparent soaps using the kit. More particularly, the present invention relates to a kit for making transparent soaps at home, which is capable of simply making a plurality of transparent soaps having various shapes at one time at home, and a method for making transparent soaps using the kit.

### BACKGROUND OF THE INVENTION

Soap is generally made by hydrolyzing vegetable oil or animal fat such as tallow or coconut oil or the like into fatty acids such as palmitic acid and stearic acid, and glycerin, neutralizing the obtained fatty acids with caustic soda or sodium carbonate and then treating fatty acid salt produced by the neutralization with various kinds of additives. Soap products, which are produced in the above mentioned manner, are classified into restroom, washing, industrial and medicated soap products in accordance with their application. The restroom soap is a soap added with an anti-oxidant, a coloring agent, a perfume, an abirritant, a builder, (for example, sodium phosphate and sodium carbonate) for increasing washing ability, and a humectant. The restroom soap has features in that it smells pleasantly owing to the addition of a perfume, has a beautiful color, has a delicate feel to the skin, and forms well.

Particularly, a transparent soap is made by adding glycerin, sugar and alcohol to such a restroom soap. The transparent soap transmits light, dissimilarly to general solid soaps, while having an excellent moisturizing property, thereby giving an improved aesthetic feeling. Because of this, the consumption of the transparent soap has increased daily.

Representative examples of methods for preparing such a transparent soap are found in several patents. U.S. Pat. No. 2,820,768 discloses a method for preparing a transparent soap by mixing a sodium soap and triethanolamine. A neutralization of excessive caustic soda and triethanolamine is carried out using fatty acids such as coconut, tallow and palm fatty acid, and castor oil. Polyol is added to the product obtained after the neutralization. Another method for preparing a transparent soap is disclosed in Japanese Patent Publication No. 81-11680. In this method, a fatty-acid soap is used as a substrate, and alcohol is used in an excessive amount for a neutralization reaction. Transparency aids such as sugar, glycerin, sorbitol, propylene glycol, and polyhydric alcohols, and solvents such as ethanol and water are heated, mixed, cooled, solidified, and then dried for several weeks, thereby forming a transparent soap. U.S. Pat. No. 3,969,259 discloses a transparent soap prepared by just adding an antibiotic ingredient to a conventional transparent soap. In this patent, Irgasan DP300 is used, which is triclosan having antibiotic ingredients. Also, besides these methods, various preparation methods of transparent soaps have been proposed.

However, these conventional methods for preparing a transparent soap are designed for massproduction. Accordingly, these methods cannot be applied to case of simply making soap at home. For example, young students have a difficulty in making soap for educational purposes at home using these methods.

### BRIEF SUMMARY OF THE INVENTION

Therefore, the present invention has been made in view of the above problems, and it is an object of the present invention to provide a kit for making transparent soaps at home and a method for making transparent soaps using the kit, which can be used to make transparent soaps having various shapes at one time at home.

It is another object of the present invention to provide a kit for making transparent soaps at home and a method for making transparent soaps using the kit so that young students can make transparent soaps easily for educational purposes.

In accordance with one aspect of the present invention, the above and other objects can be accomplished by the provision of a kit for making transparent soaps at home comprising an upper plate made of a flexible synthetic resin material and provided with a plurality of mould cavities having different shapes respectively; and a sidewall extending downward from the upper plate and formed with legs at all sides of a lower end of the sidewall, respectively, so that lower surfaces of the mould cavities are spaced apart from a supporting surface.

In accordance with another aspect of the present invention, there is provided a method for making transparent soaps having various shapes at one time at home using the kit having the structure described above, comprising the steps of:

preparing a transparent soap composition by mixing ingredients of a transparent soap;

melting the transparent soap composition to make a molten-state composition;

pouring the molten composition to the mould cavities of the kit one by one, and solidifying the composition; and

pushing the lower surfaces of the mould cavities upward, respectively, thereby extruding the solidified transparent soaps.

### BRIEF DESCRIPTION OF THE SEVERAL VIEWS OF THE DRAWINGS

The above and other objects, features and other advantages of the present invention will be more clearly understood from the following detailed description taken in conjunction with the accompanying drawings, in which:

FIG. 1 is an exploded perspective view of an example of a kit according to the present invention; and

FIG. 2 is a cross-sectional view of the kit of the present invention showing a procedure of preparing transparent soaps using the kit according to the present invention.

### DETAILED DESCRIPTION OF THE INVENTION

Now, the characteristics and advantages of the present .invention will be described in detail with reference to the annexed drawings. Also, the terms and words used in the following description and claims are defined to describe the present invention. The definition of these terms should be determined based on the whole content of this specification.

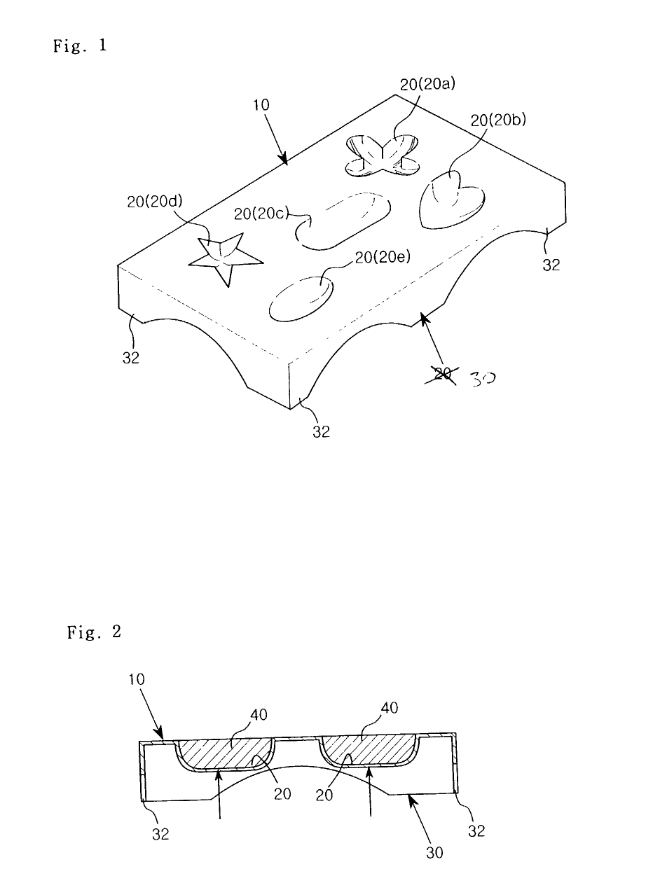

A kit for preparing transparent soaps usable as a soap dish according to the present invention is illustrated in FIG. 1. As shown in FIG. 1, the kit includes an upper plate 10, and a sidewall 30 extending downward from the entire edge of the upper plate 10.

The upper plate 10 is made of a flexible synthetic resin material. The flexible synthetic material preferably includes easily recyclable polyethylene. The upper plate 10 has a plurality of mould cavities 20 opened upward. The mould cavities 20 are respectively formed in various shapes, for example, animal and plant shapes as well as a clover 20a, a heart 20b, an obround shape 20c, a star 20d and an elliptical shape 20e, to arouse young students' curiosities.

The sidewall 30 extending downward from the entire edge of the upper plate 10 is also made of a synthetic resin material. The sidewall 30 is preferably integral with the upper plate 10. Legs 32 are respectively formed at all sides of the lower end of the sidewall 30 so that when the kit is set on a supporting surface, the lower surfaces of the mould cavities 20 are spaced apart from the supporting surface. The legs 32 may be further formed on the sides of the sidewall 30 corresponding to the long edges of the upper plate 10, respectively, to safely support the kit.

The present invention shows an example of the kit having a rectangular structure, but is not limited thereto. Although not shown, kits of diverse structures, for example, an ellipse, a circle, a lozenge and the like, can be provided. Also, any kits can be included within a scope of the present invention in so far as they are applicable to the method for making transparent soaps according to the present invention.

Now, a method for preparing transparent soaps using the above described kit for preparing transparent soaps according to the present invention will be described.

In accordance with the present invention, the preparation method includes the steps of preparing a transparent soap composition, melting the transparent soap composition, injecting the molten composition to the kit, solidifying the molten composition and taking the solidified soap out of the kit.

This method will now be described in more detail. First, various kinds of raw ingredients required for preparing transparent soaps are prepared, and mixed at a specific proportion, thereby forming a transparent soap composition. The ratio of the composition may be easily appreciated by referring to the references cited in the description of the related art and thus a detailed description thereof will be omitted herein. Also, in accordance with the present invention, since these raw ingredients are commercially available in the form of a mixed product, along with the kit of the present invention, it is unnecessary to additionally provide the raw ingredients of the transparent soap.

The transparent soap composition prepared as described above is melted by a heating means while being diluted.

The molten composition is poured into the mould cavities 20 of the kit 10 one by one and then solidified for a desired time, thereby forming transparent soaps 40 as shown in FIG. 2.

The transparent soaps 40 thus prepared are respectively extruded by pushing lower surfaces of the mould cavities 20 upward with the fingers in the direction of an arrow shown in FIG. 2. The extrusion is easily achieved because the kit according to the present invention is made of flexible synthetic resin material, so that the lower surfaces of the mould cavities 20 are easily raised.

After preparing the transparent soaps 40 as described above, the kit is used to store the prepared transparent soaps 40, or reused to prepare other transparent soaps.

As apparent from the above description, the present invention provides a kit for making transparent soaps at home and a method for making transparent soaps using the kit, which can be used to conveniently make transparent soaps having various shapes at one time at home, thereby giving an economical effect. Particularly, since young students can make transparent soaps easily using the kit and method, their scientific curiosities are stimulated.

Also, this method has an advantage in that the kit having been used in making transparent soaps can be used to store the transparent soaps.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

I claim:

1. A kit for making transparent soaps at home comprising
an upper plate made of a flexible synthetic resin material and provided with a plurality of mould cavities having different shapes respectively; and
a sidewall extending downward from the upper plate and formed with legs at all sides of a lower end of the sidewall, respectively so that lower surfaces of the mould cavities are spaced apart from a supporting surface.

2. A method for making transparent soaps at home, comprising the steps of
preparing a kit including an upper plate made of a flexible synthetic resin material and provided with a plurality of mould cavities having different shapes respectively, and a sidewall extending downward from the upper plate and formed with legs at all sides of a lower end of the sidewall, respectively, so that lower surfaces of the mould cavities are spaced apart from a supporting surface;
preparing a transparent soap composition by mixing ingredients of a transparent soap;
melting the transparent soap composition to make a molten-state composition;
pouring the molten composition to the mould cavities of the kit one by one, and solidifying the composition; and
pushing the lower surfaces of the mould cavities upward, respectively, thereby extruding the solidified transparent soaps.
